(19) United States
(12) Patent Application Publication
(10) Pub. No.: US 2009/0202581 A1
(43) Pub. Date: Aug. 13, 2009

(54) Cosmetic formulation

(76) Inventor: Nathalie Schlemer, Culver City, CA (US)

Correspondence Address: Cislo & Thomas LLP, 1333 2nd Street, Suite #500, Santa Monica, CA, 90401-4110, US

(21) Appl. No.: 12/069,855

(22) Filed: Feb. 12, 2008

(51) Int. Cl. A61K 8/99; A61Q 19/00; A61Q 19/08

(52) U.S. Cl. 424/195.16

(57) ABSTRACT

The present invention provides an improved skin care formulation for enhancing the overall quality of the skin and treating aging skin comprising cassia angustilfolia seed polysaccharide, dipeptide-4, glutamylamidoethyl indole, and kluyveromyces extract, in a cosmetically/dermatologically acceptable medium. The formulation improves skin quality by moisturizing and softening the skin, while protecting and repairing damaged sensitive skin, activating skin restructuration and stimulating collagen and keratin synthesis. Additionally, the formulation protects the skin from stress and oxidation, and reduces protein carbonylation and lipid peroxidation.

### BACKGROUND

1. Field of the Invention

The present invention relates to cosmetic compositions and particularly skin care compositions.

2. Description of the Related Art

There is always a need in the cosmetic industry for improved products which function effectively, and improve the overall quality of the skin. There are many known skin benefiting agents and skin care compositions available on the market which may combine several different agents, each having its own beneficial effect to improve overall skin care quality.

A major concern in the improvement of skin care has been treating the signs of aging, especially on the face. As such, there are many products on the market directed towards fighting various skin conditions associated with aging, such as fine lines and wrinkles, loss of skin elasticity and firmness, sagging, dryness, age spots, etc. However, as skin care needs vary from person to person, no single products suits every individual. Thus, there is always a need for improved products, especially compositions which are effective in the treatment of skin conditions associated with aging, and suitable for sensitive skin types.

### SUMMARY

The present invention provides an improved skin care formulation for enhancing the overall quality of the skin and treating aging skin comprising cassia angustilfolia seed polysaccharide, dipeptide-4, glutamylamidoethyl indole, and kluyveromyces extract, in a cosmetically/dermatologically acceptable medium. The formulation improves skin quality by moisturizing and softening the skin, while protecting and repairing damaged sensitive skin, activating skin restructuration and stimulating collagen and keratin synthesis. Additionally, the formulation protects the skin from stress and oxidation, and reduces protein carbonylation and lipid peroxidation.

### DETAILED DESCRIPTION

The detailed description set forth below is intended as a description of exemplary embodiments and is not intended to represent the only forms in which the exemplary embodiments may be constructed and/or utilized. The description sets forth the functions and the sequence of steps for constructing and/or operating the exemplary embodiments. However, it is to be understood that the same or equivalent functions and sequences may be accomplished by different exemplary methods are also intended to be encompassed within the spirit and scope of the invention.

As used herein, “safe and effective amount” means a sufficient amount of a compound, composition or other material described by this phrase to significantly induce a positive modification in the condition being treated, but low enough to avoid undue side effects (e.g., significant skin irritation or sensitization), within the scope of sound judgment of the skilled person. The safe and effective amount of the compound, composition or other material may vary with the particular skin being treated, the age and physical condition of the biological subject being treated, the severity of the condition, the duration of treatment, the nature of concurrent therapy, the specific compound, composition, or other material employed, the particular cosmetically acceptable topical carrier utilized, and the factors within the knowledge and expertise of the skilled person.

The present invention provides an improved skin care formulation for enhancing the overall quality of the skin and treating aging skin comprising cassia angustilfolia seed polysaccharide, dipeptide-4, glutamylamidoethyl indole, and kluyveromyces extract, in a cosmetically/dermatologically acceptable medium. The formulation improves skin quality by moisturizing and softening the skin, while protecting and repairing damaged sensitive skin, activating skin restructuration and stimulating collagen and keratin synthesis. Additionally, the formulation protects the skin from stress and oxidation, and reduces protein carbonylation and lipid peroxidation. It is preferably formulated in a serum or other acceptable carrier, such as a cream, lotion, gel, cream-gel, vesicle or capsule dispersion, emulsion, milk, etc.

The formulation includes cassia angustilfolia seed polysaccharide which is a Beta-Glucan that comes from the fruit seeds of the Indian plant Cassia Angustifolia and may be commercially available from COGNIS under the trade name INDINYL™ CA. This component has film forming capabilities and helps to provide sustained moisturizing on the surface of the skin, thus softening and improving skin complexion and radiance. It further protects and repairs sensitive, rough dry skin. The formulation preferably includes INDINYL™ CA at a concentration of up to 10%, and more preferably at 2.5%.

The formulation further includes dipeptide-4, which is a Glutathione-biomimetic synthetic peptide, available from MMP Inc. under the trade name QUINTESCINE™ (dipeptide-4, butylene glycol). It is an anti-stress anti-oxidant peptide, that protects against glycation damage, and prevents loss of skin resiliency. It further reduces protein carbonylation and lipid peroxidation, and improves DNA protection. The formulation of the present invention preferably includes QUINTESCINE™ at a concentration of between 0.1 to 10%, and more preferably at around 1%.

The formulation further includes glutamylamidoethyl indole, which is a neuroactive, available from EXSYMOL or BIOSIL, under the name GLISTIN™. This component is an anti-oxidant, anti-stress agent which provides cosmetic neuroprotection and stabilizes cells neural network for healthier cells. The formulation of the present invention preferably includes GLISTIN™ at a concentration of between 0.1 to 10%, and more preferably at around 1%.

The formulation further includes hydrolyzed kluyveromyces extract available from MMP under the name NEOVITYLINE. This active protects and repairs the skin, activates skin restructuration, and stimulates collagen and keratin synthesis. The formulation includes NEOVITYLINE at a concentration of between 0.1 to 10%, and more preferably at around 2%.

The following is an example for the preparation of a serum comprising the ingredients listed in the table below, in accordance with a preferred embodiment of the invention.

| Sequence | Trade | | | % Active | | |
|---|---|---|---|---|---|---|
| No. | Name | INCI Name | % Wt | (Breakdown) | Function | Mfg |
| 1-01 | Deionized | Water (Aqua) | 37.15% | | Diluent | |
| | Water | | | | | |
| 1-02 | Keltrol | Xanthan Gum | 20.00% | 1% Xanthan | Thickener, | Kelco |
| | (1%) | | | Gum, | Emulsion | |
| | | | | 99% Water | Stabilizer | |
| 1-03 | Dissolvine | Disodium EDTA | 0.10% | | Chelating Agent | Akzo Nobel |
| | Na2S | | | | | |
| 1-04 | Botanistat | Phenoxyethanol | 1.00% | Phenoxyethanol | Preservative | Botanigenics |
| | PF-64 | (and) Caprylyl | | 50%, Capryl | | |
| | | Glycol (and) | | Glycol 20-30%, | | |
| | | Ethyhexhyl | | Ethylhexyl | | |
| | | glycerin (and) | | glycerin 10-20%, | | |
| | | Hexylene Glycol | | Hexylene Glycol | | |
| | | | | 10-20% | | |
| 2-01 | Emulium | Cetyl Alcohol | 4.00% | 35%, 35%, 16%, | Emulsifier | Gattefosse |
| | Delta | (and) Glyceryl | | 7%, 7% | | |
| | | Stearate (and) | | | | |
| | | PEG-75 Stearate | | | | |
| | | (and Ceterth-20 | | | | |
| | | (and) Steareth-20 | | | | |
| 2-02 | Liponate | Pentaerythrityl | 3.00% | | Emollient | Lipo |
| | PE-810 | Tetracaprylate/ | | | | Chemicals |
| | | Tetracaprate | | | | |
| 2-03 | Dow | Dimethicone | 8.00% | | Emollient | Dow |
| | Corning | | | | | Chemicals |
| | 200/100cs | | | | | |
| 2-04 | Lipocol C | Cethyl Alcohol | 1.00% | | Emollient, | Lipo |
| | | | | | Emulsifier, | Chemicals |
| | | | | | Opacifier | |
| 2-05 | Stearic Acid | Stearic Acid | 0.75% | | Thickener, | Lipo |
| | | | | | Emulsion | Chemicals |
| | | | | | Stabilizer | |
| 2-06 | Softisan 100 | Hydrogenated | 2.00% | | Emollient | Sasol North |
| | | Coco-Gylcerides | | | | America |
| 2-07 | Lipopeg | PEG-100 Stearate | 1.50% | | Emulsifier | Lipo |
| | 100-S | | | | | Chemicals |
| 3-01 | Dow | Cyclopenta | 11.00% | 60% Cyclopenta | Mattifying Agent | Dow |
| | Corning | siloxane (and) | | siloxane, 10-30% | | Chemicals |
| | 9040 | Dimethicone | | Dimethicone | | |
| | Silicone | Crosspolymer | | Crosspolymer | | |
| | Elastomer | | | | | |
| 3-02 | Dow | Cyclopenta | 4.00% | 60%, 40% | Emollient | Dow |
| | Corning 345 | Siloxane, Cyclo | | | | Chemicals |
| | Fluid | Hexasiloxane | | | | |
| 4-01 | Indinyl CA | Water (Aqua) | 2.50% | Water > 50%, | An exotic beta- | Cognis |
| | LS8998 | (and) Cassia | | Cassia | glucan that | |
| | | Angustifolia Seed | | Angustifolia | moisturizes and | |
| | | Polysaccharide | | Seed, | softens skin and | |
| | | | | Polysaccaharide | protects and | |
| | | | | 1-5% | repairs sensitive | |
| | | | | | skin | |
| 4-02 | Quintescine | Water (Aqua) | 1.00% | Water 68.995%, | Anti-stress, | MMP |
| | | (and) Butylene | | Butylene Gylcol | Antioxidant | |
| | | Glycol (and) | | 30%, Dipeptide | Peptide, reduces | |
| | | Dipeptide-4 | | 0.005% | protein | |
| | | | | | carbonylation, | |
| | | | | | improves DNA | |
| | | | | | protection and | |
| | | | | | decreases lipid | |
| | | | | | peroxidation | |
| 4-03 | Glistin | Glutamylamidoethyl | 1.00% | | Cosmetic Neuro- | Exsymol |
| | | Indole | | | protection, | |
| | | | | | antioxidant effect, | |
| | | | | | anti-stress, | |
| | | | | | stabilizes cells | |
| | | | | | neural network for | |
| | | | | | healthy cell | |
| | | | | | growth | |
| 4-04 | Neovityline | Water (Aqua) | 2.00% | Water73%, | Protects and | MMP |
| | | (and) Butylene | | Butylene Glycol | repairs the skin, | |
| | | Glycol (and) | | 25%, Hydrolyzed | activates skin | |
| | | Hydrolyzed | | Kluveromyces | restructuration, | |
| | | Kluyveromyces | | Extract 2% | stimulates collagen | |
| | | Extract | | | and Keratin | |
| | | | | | synthesis | |

The serum is prepared according to the following procedure:

In a main kettle, combine Sequences 1-01 through 1-04 and heat to 80° C. with propeller mixing to form a Sequence 1 mixture. Combine Sequences 2-01 through 2-07 and heat to 80° C., to form a Sequence 2 mixture. Mix until clear and uniform. Combine Sequences 3-01 through 3-02 and mix until uniform to form a Sequence 3 mixture. Add the Sequence 3 mixture to the Sequence 2 mixture. Add the combined Sequence 2 and 3 mixtures to the Sequence 1 mixture with propeller mixing forming the main batch. Homogenize the main batch for approximately 3 minutes. Begin cooling. At approximately 30°-35° C. add Sequences 4-01 through 4-04 one at a time to the main batch, mixing well after each addition. Cool to 25° C.

In closing, it is to be understood that the exemplary embodiments described herein are illustrative of the principles of the present invention. Other modifications that may be employed are within the scope of the invention. Thus, by way of example, but not of limitation, alternative configurations may be utilized in accordance with the teachings herein. Accordingly, the description is illustrative and not meant to be a limitation thereof.

What is claimed is:

1. A cosmetic moisturizing and anti-aging composition comprising:
approximately between 0.1 to 10% Indinyl;
approximately between 0.1 to 10% Quintescine;
approximately between 0.1 to 10% Glistin;
approximately between 0.1 to 10% Neovityline;
thickeners and emulsion stabilizers comprising xanthan gum, and stearic Acid;
a chelating agent comprising EDTA;
preservatives comprising Phenoxyethanol, Caprylyl Glycol, Ethyhexhyl glycerin, and Hexylene Glycol;
emulsifiers comprising Cetyl Alcohol, Glyceryl Stearate, PEG-75 Stearate, Ceterth-20, Steareth-20, and PEG-100 Stearate;
emollients comprising Pentaerythrityl Tetracaprylate/Tetracaprate, Dimethicone, Cethyl Alcohol, Hydrogenated Coco-Gylcerides, CyclopentaSiloxane, and CycloHexasiloxane; and
matifying agents comprising Cyclopentasiloxane and Dimethicone Crosspolymer, whereas said composition for improving the appearance of aging skin.

2. A cosmetic moisturizing and anti-aging composition comprising:
angustilfolia seed polysaccharide;
dipeptide-4;
glutamylamidoethyl indole; and
hydrolyzed kluyveromyces extract
in a in a cosmetically/dermatologically acceptable medium, wherein said composition protects and repairs damaged skin, activates restructuration and stimulates collagen synthesis of the skin.

3. The composition of claim 2 further comprising hydrogenated coco-glycerides, cyclopentasioxane, dimethicone crosspolymer xanthan gum, disodium EDTA, cetyl alcohol, glyceryl stearate, and stearic acid.

4. The composition of claim 2 wherein said cosmetically/dermatologically acceptable medium is a serum.

5. The composition of claim 2 wherein said angustilfolia seed polysaccharide is available under the trade name INDINYL™, said composition comprising up to 10% INDINYL™.

6. The composition of claim 2 wherein said dpeptide-4 is available under the trade name QUINTESCINE™, said composition comprising approximately 0.1 to 10% QUINTESCINE™.

7. The composition of claim 2 wherein said glutamylamidoethyl indole is available under the trade name GLISTIN™, said composition comprising approximately 0.1 to 10% GLISTIN™.

8. The composition of claim 2 wherein said hydrolyzed kluyveromyces extract is available under the trade name NEOVITYLIME™, said composition comprising approximately 0.1 to 10% NEOVITYLIME™.
